(19) United States
(12) Patent Application Publication
(10) Pub. No.: US 2003/0148260 A1
(43) Pub. Date: Aug. 7, 2003

(54) Method of diagnosing colorectal adenomas and cancer using proton maggnetic resonance spectroscopy

(76) Inventors: Bernard Levin, Houston, TX (US); Ian C.P. Smith, Winnipeg (CA); Rajmund Lewis Somoraji, Headingley (CA); Constance M. Johnson, Bellaire, TX (US); Tedros Bezabeh, Winnipeg (CA); Charles Noah Bernstein, Winnipeg (CA)

Correspondence Address: George A. Seaby, Seaby & ASSOCIATES, 603-880 Wellington Street, Ottawa, ON, K1R 6K7, CA

(21) Appl. No.: 10/359,088

(22) Filed: Feb. 6, 2003

Related U.S. Application Data
(63) Continuation of application No. PCT/CA01/01129, filed on Aug. 7, 2001.
(60) Provisional application No. 60/411,783, filed on Sep. 19, 2002.

(51) Int. Cl. C12Q 1/00; G01N 33/574

(52) U.S. Cl. 435/4; 435/7.23

(57) ABSTRACT

One dimensional proton magnetic resonance spectroscopy of human stool can be used as non-invasive method of detecting the presence of colorectal cancer and/or clinically significant adenomas. The spectrum of a patient's stool is compared with that of stool from non-cancerous subjects, observed differences in spectra being indicative of cancer and/or clinically significant adenomas. In a preferred method, the stool sample is mixed with a buffer, the resulting suspension is centrifuged and the supernatant is subjected to magnetic resonance spectroscopy.

This application is a continuation-in-part of PCT Application CA01/01129, filed Aug. 7, 2001 which claims priority to U.S. provisional application No. 60/233,994, filed Aug. 9, 2000. This application also claims priority to U.S. provisional application No. 60/411,783, filed Sep. 19, 2002.

This invention relates to a method of detecting colorectal adenomas and cancer, and in particular to a method of detecting such adenomas and cancer using proton magnetic resonance spectroscopy.

Colorectal cancer is one of the most common cancers in the U.S.A. and Canada accounting for approximately 146,000 new cases in 1999. The lifetime risk that an individual in North America will develop colorectal cancer is about 5-6%. Symptoms associated with colorectal cancer, including blood in the stool, anemia, abdominal pain and alteration of bowel habits often become apparent only when the disease has advanced significantly. It is well known that prognosis for a patient depends largely on the stage of the disease at the time of diagnosis. In fact, whereas the five-year survival for a patient whose colorectal cancer is detected at an early stage is 92%, survival decreases to about 60% in patients with regional spread, and to about 6% in those with distant metastases. Accordingly, it is important to detect the precursor adenomas and cancer as early as possible to increase the chances of successful therapeutic intervention.

Screening for a disease requires that the disease be prevalent in a large segment of the population and that early detection of the disease decreases mortality and improves quality of life. Colorectal cancer meets these requirements (Mandel J S, Church T R, Ederer F, Bond J H, Colorectal cancer mortality: effectiveness of biennial screening for fecal occult blood. J Natl Cancer Inst 1999; 91:434-437 and Mandel J S, Bond J H, Church T R, Snover D C, Bradley G M, Schuman L H, Ederer F, Reducing mortality from colorectal cancer by screening for fecal occult blood. Minnesota colon cancer control study, N Eng J Med 1993;328; 1365-1371) and, thus, is an ideal candidate for such a program. The natural history of colorectal cancer, namely the progression from adenoma to adenocarcinoma occurring over a number of years (5-15), also makes it a suitable target. The cost benefit analysis for the early detection of colorectal cancer has also been shown to be favourable (Bolin, T D. Cost benefit of early diagnosis of colorectal cancer. Scand J Gastroenterol 1996; 31 Suppl 220:142-146).

The screening technique itself also has to meet a series of criteria, such as, high sensitivity and specificity, low cost, safety and simplicity. Currently, digital rectal examination (DRE), fecal occult blood test (FOBT), barium enema and direct colon visualization (sigmoidoscopy and colonoscopy) are used for this purpose.

DRE involves examining the rectum using a finger. This method detects cancers that can be palpated and are within reach of the finger. A negative DRE provides little reassurance that a patient is free of cancer, because fewer than 10% of colorectal cancers can be palpated by the examining finger.

FOBT detects hidden blood in the stool by chemical means. Although the least expensive and the simplest, the FOBT method has low sensitivity, moderate specificity and is usually not good for early detection. According to available data, a major drawback of this technique is that more than half of the cancers discovered by this method followed by x-ray or endoscopy are usually beyond the limit of early staging. A false positive rate of 10-12% is expected when the patients tested are on an unrestricted diet. Estimates of the positive predictive value range from 2.2 to 50%. The guaiac tests have a very low sensitivity, generally around 50% (Ransohoff D F, Lang Calif., Screening for colorectal cancer with the fecal occult blood test; a background paper. Ann Intern Med 1997; 126:811-822). The use of FOBT is based on the assumption that colorectal cancers are associated with bleeding. However, some colorectal cancers bleed intermittently and others not at all.

A barium enema involves an x-ray of the bowel using a contrast agent. The enema can be a single or double contrast. The main radiologic signs of malignancy include muscosal disruption, abrupt cut-off and shouldering and localized lesions with sharp demarcations from uninvolved areas. The estimated sensitivity of double contrast barium enema for cancer and large polyps is only about 65-75% and even lower for small adenomas. Despite its better diagnostic yield, double contrast barium enema has a false-negative rate of 2-18%. Moreover, the method involves exposure to radiation, the repeated use of which may not be safe. Perforation from barium enema is extremely uncommon, but when it happens it is can be fatal or leads to serious long term problems as a result of barium spillage into the abdominal cavity.

A variety of instruments (collectively called endoscopes) are used for examining the bowel. Endoscopes can be rigid or flexible with varying lengths. Flexible sigmoidoscopes are 60 cm long. A colonoscope is a 130-160 cm flexible viewing instrument for examining the entire colon. Biopsies are taken from suspicious looking areas while viewing the colon through the endoscope. The flexible sigmoidoscopy examination is limited to the left side of the colon and rectum. At least ⅓ of neoplastic tumors occur in areas proximal to the splenic flexure that are inaccessible by sigmoidoscopy. Colonoscopy has a high sensitivity, and remains the gold standard for visualization of the colon and the detection of neoplastic abnormalities. However, it is invasive, quite expensive, and exposes the subject to risks of bowel perforation.

Magnetic resonance spectroscopy (MRS) is a technique that has the potential to detect small and early biochemical changes associated with disease processes, and has been proven to be useful in the study of tissue biopsies from cancer patients (Smith I. C. P, Bezabeh T, Tissue NMR Ex Vivo.In: Young I R, ed Methods in Biomedical magnetic resonance imaging and spectroscopy, Chicester, U K; Wiley, 2000:891-7). It is particularly useful to detect small, mobile chemical species in a given biological sample that are of diagnostic interest. Obtaining tissue biopsies for such an examination, however, usually involves an invasive procedure.

There are a number of currently available methods for detecting cancer in its stages. Biophysical methods such as conventional X-rays, nuclear medicine, rectilinear scanners, ultrasound, CAT and MRI all play an important role in early detection and treatment of cancer. Clinical laboratory testing for tumor markers can also be used as an aid in early cancer detection. Tumor marker tests measure either tumor-associated antigens or other substances present in cancer patients which aid in diagnosis, staging, disease progression, monitoring response to therapy and detection of recurrent disease. Unfortunately, most tumor marker tests do not possess sufficient specificity to be used as screening tools in a cost-effective manner. Even highly specific tests suffer from poor predictive value, because the prevalence of a particular cancer is relatively low in the general population. The majority of available tumor marker tests are not useful in diagnosing cancer in symptomatic patients because elevated levels of markers are also seen in a variety of benign diseases. The main clinical value of tumor markers is in tumor staging, monitoring therapeutic responses, predicting patient outcomes and detecting recurrence of cancer.

U.S. Pat. Nos. 4,912,050 and 4,918,021, issued to E. T. Fossel on Mar. 27, 1990 and Apr. 17, 1990, respectively disclose a technique for detecting cancer by proton nuclear magnetic resonance (NMR) of blood, blood serum or blood plasma. U.S. Pat. No. 5,261,405, issued to the same inventor on Nov. 16, 1993 describes an apparatus and method for automating the process.

U.S. Pat. No. 5,318,031, issued to Mounfford et al on Jun. 7, 1997 describes a method for determining chemical states of living animal or human tissue using one-dimensional NMR and 2D-COSY (two-dimensional correlation) NMR spectroscopy, and comparing measured values to reference measurements of normal, abnormal and transitions state tissue.

C. L. Lean et al (Magn. Reson Med 20:306-311, 1991; Biochemistry 3:11095-11105, 1992 and Magn Reson Med 30:525-533, 1992) describe the use of magnetic resonance spectroscopy to examine colon cells and tissue specimens.

However, a need still exists for an inexpensive, non-invasive method of detecting colorectal cancer and colorectal adenomas. The object of the present invention is to provide a relatively simple, non-invasive method of detecting colorectal adenomas and cancer which meets the above defined criteria of high sensitivity and specificity, low cost and safety.

Accordingly, the invention relates to a method of detecting the presence of colorectal adenomas and colorectal cancer in a patient comprising the steps of subjecting a stool sample from the patient to magnetic resonance spectroscopy; and comparing the resulting spectrum with the magnetic resonance spectra of stool from non-cancerous subjects, with observed differences in spectra being indicative of cancer or clinically significant adenomas.

In a preferred embodiment of the method, the stool sample is mixed with a buffer to produce a suspension of stool sample, the suspension is centrifuged to yield a supernatant sample, the supernatant sample is subjected to magnetic resonance spectroscopy, and the resulting spectrum is compared with magnetic resonance spectra of stool from non-cancerous subjects.

The performing of spectral analysis on human stool offers a significant advantage over other methods, because the collection of the specimen is non-invasive and presents no risk to the patient. Moreover, no special processing of the sample is required prior to analysis.

While the inventors reported earlier that the use of 2D-COSY spectroscopy is preferred, further testing has revealed that one-dimensional proton magnetic resonance spectroscopy is preferred. In 1 D MRS, the early onset of colorectal cancer is determined by the detection of spectral profiles/features characteristics of colonic neoplasia by performing multivariate analysis on one-dimensional proton magnetic resonance spectra of human stool.

### METHOD

One hundred and twenty-two subjects, who were scheduled for colonoscopy or surgery were recruited to donate a single sample of stool. Table 1 provides a breakdown of the cases.

TABLE 1
(Breakdown of subjects recruited)

| Cases | |
|---|---|
| Colorectal cancer | 34 |
| Normal | 50 |
| Adenomatous Polyps | 38 |

The group referred to as “Normal” includes some subjects with colonic conditions/abnormalities that are non-neoplastic. Examples include diverticulosis, hyperplastic polyps and internal hemorrhoids. Specimens from subjects with inflammatory bowel disease are not included in the analysis.

Stool samples were collected at the University of Texas M. D. Anderson Cancer Center; University of Manitoba, Health Sciences Centre; University of Chicago; and University of Toronto, Mount Sinai Hospital. The samples were kept frozen in the patients' refrigerators for an average of 24-48 hours prior to their delivery to the hospital in small ice chests (mailers). They were then stored in a −70° C. freezer until being shipped to the National Research Council Institute for Biodiagnostics, Winnipeg, Canada. All samples were shipped in dry ice and kept frozen at −70° C. until the time of the experiment. There was no significant difference in the lengths of time for which the samples were kept frozen.

Sample Preparation (I)

For MRS experiments, samples were thawed and homogenized, and an aliquot portion was taken and suspended in PBS/D₂O buffer. The suspension was then put inside a capillary tube (filled to approximately one-third of its volume) with one end closed with a Teflon (trademark for polytetrafluoroethylene) plug. This was then put in a standard 5 mm MR tube containing p-amino benzoic acid (PABA) that served as a chemical shift reference.

MRS Experiments (I)

All experiments were performed at 360 MHz (Bruker Instruments) at 25° C. with presaturation of the water signal. The 1 D acquisition parameters include 90° pulse at 9 ps; number of scans, NS=256; recycle delay, RD=2.41 s; time domain data points, TD=4K; spectral width, SW=5000 Hz.

Data Processing (I)

The 0.5-2.5 ppm region (300 data points) of each spectrum is used for the analysis, to minimize the influence of spectral artifacts created by suppression of the water peak at 4.6 ppm and the resonance at 3.7 ppm due to polyethylene glycol contained in the colon lavage solution used to flush the colon as part of the preparation for colonoscopy and for surgery in some patients. All Spectra are aligned on a reference peak. Each magnitude spectrum is normalized by dividing every data point by the total spectral area or by rank ordering the spectral intensities.

The classification strategy used has been developed specifically to deal with the discrimination of spectra of biomedical origin. The strategy comprises three stages. The first stage is a preprocessing step, found to be essential for reliable classification. It consists of selecting from the spectra a few maximally discriminatory subregions, using an optimal region selection (ORS) algorithm, based on a genetic algorithm (GA)-driven optimization method (Nikulin A E, Dolenko B, Bezabeh T, Somorjai R L, NMR in Biomedicine 11, 209-217 (1998), “Near-optimal region selection for feature space reduction: Novel preprocessing methods for classifying MR spectra” Bezabeh, T et al, The use of ¹H Magnetic Resonance Spectroscopy in Inflammatory Bowel Disease: Distinguishing Ulcerative Colotis From Crohn's Disease, Am. J. Gastroenterol 2001; 96: 442-448 and Somorjai, R. L. et al, Distinguishing Normal from Rejecting Renal Allographs: Application of a Three-Stage Classification Strategy to MR and IR Spectra of Urine, Vibrational Spectroscopy 28 (1) 97-102 (2002)). For reliability of classification, the number of these subregions should be an order of magnitude smaller than the number of samples to be classified. To avoid the overly optimistic classification results that a straight resubstitution approach would give, the inventors have developed a cross-validation method, using a bootstrap methodology.

The bootstrap method repeatedly partitions (with replacement) the data into approximately equal sized random training and test subsets. For each of the random training subsets an optimal classifier is found, and its accuracy validated on the random test subset. The process is repeated a number of times (1000 times at the less critical ORS preprocessing stage, 5000 times for the final classifier). Once the optimal subregions are identified, the second stage finds the ultimate classifier as the weighted average of the classifier coefficients of the 5000 individual component classifiers. This approach effectively uses all n samples. A standard multivariate statistical method, Linear Discriminant Analysis (LDA) is the choice for all classifiers, at all stages, because of its speed and robustness. The concept of crispness of a classifier is also used because the inventors' classifiers produce class probabilities. The inventors call a 2-class classification of a sample crisp if the class assignment probability for that sample is >75%.

For difficult classification problems, a third stage consists of combining the outcomes of several classifiers via aggregation methods (computerized consensus diagnosis, CCD) into an overall classifier that is more reliable and accurate than the individual classifiers. The particular classifier aggregation used by the inventors is Wolpert's Stacked Generalizer (WSG). WSG uses the output class probabilities obtained by the individual classifiers as input features to the ultimate classifier. For 2-class problems the number of features is 1 per classifier (with K independent classifiers this gives K probabilities as input features). The overall classification quality is generally higher. The crispness of the classifier is invariably greater. This is important in a clinical environment because fewer patients will have to be re-examined.

Results

There were notable differences between the COSY spectra of stool specimens from normal subjects and those with colorectal carcinoma. Of particular interest is the appearance of a crosspeak at 1.3-4.3 (attributed to the methyl-methine couplings of bound fucose) that was suggested to serve as a marker for the presence of colorectal cancer (C. L. Lean et al, Magn Reson Med 20:306-311, 1991).

Based on the presence or absence of this crosspeak, specimens were identified as being positive or negative for malignancy. The sensitivity, specificity and positive predictive value (PPV) for the analysis are indicated in Table 2. The reported sensitivity of FOBT extends from 24-78% (Young, G. P. et al, Clinical Methods for Early Detection: Basis, Use and Evaluation; Chapter 13, pp 242-270 in Prevention and Early Detection of Colorectal Cancer, ed Young, G. P. et al, London, 1996). These values are lower for adenomatous polyps. The 2D COSY MR results yield a much higher accuracy than those of the FOBT technique. The 2D results are from earlier work by the inventors, and are presented for comparison purposes only.

The 2D COSY technique, which has reasonable accuracy and casts light on the nature of the diagnostic compound, has the disadvantage of long acquisition time (one to several hours). Furthermore, the absence of a peak can have sources other than the absence of the compound. The inventors, therefore turned to multivariate analysis of the corresponding one-dimensional spectra, a method which has been extremely successful for cancer biopsies (Somorjai, R. et al, J. Mag. Reson. Imaging 6, 437 (1996)). These spectra require only minutes for acquisition, provide a wide variety of data points, and can be analyzed automatically once the diagnostic algorithm has been authenticated.

The results of the two-dimensional and the one-dimensional approach are shown in Table 2. The 1 D results demonstrate a higher sensitivity and specificity than given by the two-dimensional approach. The values for the 2D COSY results drop significantly when polyps are included with the cancers, as discussed above.

TABLE 2

| | |
|---|---|
| 2D COSY | 1D MRS |

| | | | | | | |
|---|---|---|---|---|---|---|
| Sens. | Spec. | PPV | Sens. | Spec. | PPV | % Crispness |

| | | | | | | | |
|---|---|---|---|---|---|---|---|
| Cancer vs. Normal | 83 | 85 | 91 | | | | |
| Cancer + Adenomatous | | | | | | | |
| Polyps vs. Normal | 77 | 85 | 94 | 98.7 | 96.4 | 97.4 | 99.2 |

a. 2D COSY results for 69 patients
b. 1D MRS results for 122 patients

In a further series of experiments, a centrifuge was used to prepare supernatant samples from patients' stool. The stool was homogenized and then spun down, the rationale being that the diagnostic markers in the sample are more than likely small, water-soluble species. The supernatant is believed to contain the diagnostic information and spectra are improved by removal of the solid matter.

Sample Preparation (II)

Specifically, homogenated raw stool sample was removed from the −70° C. freezer and placed in a biohood (at room temperature) for approximately 10-15 minutes. The sample did not thaw completely in this time, but gets slightly softer and a small piece could be chipped off easily. About 2 grams of specimen was taken using a spatula and placed in a small glass vial. After a complete thaw, 6 ml of PBS/D₂O buffer was added to the vial, making the ratio of buffer to stool approximately 3:1. The suspension was then mixed (vortexed) for 2 minutes at a speed of 5 (using Vortex-2 Genie). The suspension was poured into multiple 1.5 ml centrifuge tubes and centrifuged for 3 minutes on a bench top centrifuge unit (Biofuge Pico) at 3200 RPM. The supernatant was then separated carefully and collected in 3-4 cryovials and frozen in a −70° C. freezer.

After a freezing period of at least 12 hours, vials were taken out and placed in a biohood. After a sample was completed thawed, 600 μl of it was taken and put in a 5 mm NMR tube (which already contains 50 μl of TSP/D₂O). The TSP was used as a chemical shift reference (0 ppm) and for alignment of spectra during data preprocessing.

MRS Experiments (II)

All samples were run on an Avance 360 Spectrometer (Bruker Instruments) with no spinning. A standard was run every morning before the actual samples to verify that the spectrometer was working properly. Once this was done, the sample was then inserted in the magnet and the time noted. The temperature was set to 298° K. and lock performed on the deuterium signal (lock power=−20 dB, phase=113.5, gain=100). Manual shimming was performed concentrating on Z, Z², Z³, X and Y. The probehead was then tuned and matched. A new experiment was written for each sample and then the acquisition parameters were set up. Water suppression was performed by the use of the presaturation technique. The line width and O1 (water frequency) were noted for each experiment.

The acquisition parameters include: pulse program Zgpr, temperature, TE=298° K.; transmitter frequency, SF01=360.3316979 MHz; number of scans, NS=32; dummy scans, DS=2; 90 degree pulse width, P1=9 psec; presaturation power level, PL9=40 dB; time domain data points, TD=16K; size of real spectrum, SI=16K; interscan delay, Dl=3.0 sec; spectral width, SW=4990.02 Hz; acquisition time, AQ=1.64; delay time, D9=60 ms.

Data Processing (II)

A total of 271 samples (MR spectra) have been fully analysed to date using the three stage statistical classification strategy. Two-hundred and forty-five of these (147 normal, 98 cancer and/or adenomas) were used (in the training set) to develop the classifier. The overall accuracy on the training data (including fuzzy classifications) was 87%. The overall accuracy on the validation set (n=26; 11 normal and 15 cancer/polyps) was 88.5%. The sensitivity was 93.3% and the specificity was 81.8%.

### CONCLUSION

The results show that 1 D MRS can be used as a screening tool for colorectal carcinoma and adenoma.

We claim:

1. A method of detecting the presence of colorectal adenomas and colorectal cancer in a patient comprising the steps of subjecting a stool sample from the patient to one-dimensional proton magnetic resonance spectroscopy; and subjecting the resulting spectrum to multivariate analysis in which the spectrum is compared with the magnetic resonance spectra of stool from non-cancerous subjects, observed differences in spectra being indicative of cancer or clinically significant adenomas.

2. A method according to claim 1, wherein the stool sample is subjected to 360 MHz magnetic resonance spectroscopy.

3. A method of detecting the presence of colorectal adenomas and colorectal cancer in a patient comprising the steps of collecting a sample of the patient's stool; subjecting the sample to one-dimensional proton magnetic resonance spectroscopy; and subjecting the resulting spectrum to multivariate analysis in which the spectrum is compared with the magnetic resonance spectra of stool from non-cancerous subjects, observed differences in spectra being indicative of cancer or clinically significant adenomas.

4. A method according to claim 3, wherein the stool sample is subjected to 360 MHz magnetic resonance spectroscopy.

5. A method according to claim 2, including the steps of selecting from the spectra resulting from the one-dimensional magnetic resonance spectroscopy maximally discriminatory subregions; repeatedly partitioning the data into approximately equal sized random training and test subsets, finding an optimal classifier for each random training subset and validating the accuracy of the optimal classifier on the random test subset; and determining the ultimate classifier as the weighted average of the classifier coefficients of a large number of individual component classifiers.

6. A method according to claim 1, wherein a liquid suspension of the stool sample is subjected to spectroscopy.

7. A method according to claim 3, including the step of forming a liquid suspension of the stool sample before subjecting the sample to spectroscopy.

8. A method according to claim 7, wherein the liquid suspension is centrifuged; supernatant resulting from centrifuging is separated; and the supernant is subjected to spectroscopy.

9. A method of detecting the presence of colorectal adenomas and colorectal cancer in a patient comprising the step of mixing a stool sample from the patient with a buffer to form a suspension; centrifuging the suspension to yield a supernatant sample; subjecting the supernatant sample to magnetic resonance spectroscopy; and comparing the resulting spectrum with the magnetic resonance spectra of stool from non-cancerous subjects, observed differences in spectra being indicative of cancer or clinically significant adenomas.
